(19) United States
(12) Patent Application Publication
(10) Pub. No.: US 2002/0155071 A1
(43) Pub. Date: Oct. 24, 2002

(54) Accelerated method and instrumentation for whitening teeth

(76) Inventor: Gerald McLaughlin, Port Jefferson Station, NY (US)

Correspondence Address: KLARQUIST SPARKMAN, LLP, 121 SW SALMON STREET, SUITE 1600, PORTLAND, OR, 97204, US

(21) Appl. No.: 10/119,863

(22) Filed: Apr. 9, 2002

Related U.S. Application Data
(63) Continuation-in-part of application No. 09/531,908, filed on Mar. 21, 2000, now abandoned.
Application No. 09/531,908 is a continuation of application No. 09/045,489, filed on Mar. 20, 1998, now Pat. No. 6,108,850.
(60) Provisional application No. 60/048,423, filed on Jun. 3, 1997.

(51) Int. Cl. A61K 7/18; A61K 7/20; A61C 5/00

(52) U.S. Cl. 424/53; 433/215; 424/52

(57) ABSTRACT

A composition for whitening teeth, using a bleaching compound and a catalytic activator, where the catalytic activator catalyzes the reaction of a significant portion of the bleaching compound within a short period of time. A method for whitening teeth by providing a bleaching compound, providing a catalytic agent, and combining the bleaching agent with the catalytic agent so that a reaction of a significant portion of the bleaching agent occurs in a short period of time is also disclosed. A device for whitening teeth, consisting of a toothbrush where at least one of the bristles contains a catalytic activator is also provided. The handle of this device has a reservoir for a bleaching compound and a means for dispensing the bleaching compound. Another device for whitening teeth, consisting of a toothbrush with a catalytic activator capable of catalyzing the reaction of a bleaching compound applied to the head of the device, is further provided.

### PRIORITY CLAIM

This application is a continuation-in-part of U.S. application Ser. No. 09/531,908 filed Mar. 21, 2000, which is a continuation of U.S. application Ser. No. 09/045,489 filed Mar. 20, 1998, which claims priority from U.S. Provisional Application Serial No. 60/048,423 filed Jun. 3, 1997, all of which are incorporated herein by reference.

### FIELD OF THE DISCLOSURE

The present disclosure relates to the field of dentistry, and specifically to the whitening of teeth.

### BACKGROUND OF THE DISCLOSURE

Teeth generally become more darkly pigmented with age and exposure to such materials as tea and coffee, and it has long been a goal of dentistry to provide a means to safely and effectively reverse this darkening process. Historically there are two approaches to the problem. The first involves removing pigmentation that has adhered onto the surface of the teeth. This is commonly achieved through the use of abrasives, sometimes augmented with solvents. While rapidly effective, these techniques have the disadvantage of only being able to remove extrinsic stains, leaving all internal pigmentation unchanged. Thus the whitening effect is extremely limited.

A more recent innovation involves a method of using oxidizing agents to penetrate into the tooth structure and bleach out the undesired pigmentation. The active agents are usually either weak solutions of hydrogen peroxide or carbamide peroxide, which is more stable than hydrogen peroxide.

While effective on both extrinsic and intrinsic discolorations, one major problem encountered with this second approach is the enormous amount of treatment time needed to gain adequate penetration of the tooth structure by the whitening agent. At present, the method of application of the whitening agent utilizes either custom or stock trays that are shaped to hold the bleaching agent against the teeth to be whitened. These trays are then filled with the peroxide, and worn for long periods of time, sometimes even overnight. After a series of lengthy treatments, the teeth will usually begin to show the desired whitening effect. The length of these treatments can be discouraging and increases the cost. It is therefore desirable to find a method to more rapidly whiten the teeth.

### SUMMARY OF THE DISCLOSURE

In one disclosed embodiment, there is provided a composition for whitening teeth, using an effective amount of a bleaching compound and a catalytic activator, where the catalytic activator catalyzes the reaction of a significant portion of the bleaching compound within a short period of time, for example, about ten minutes.

In a second disclosed embodiment, there is provided a method for whitening teeth that comprises providing a bleaching compound, providing a catalytic agent, and combining the bleaching agent with the catalytic agent so that a reaction of a significant portion of the bleaching agent occurs within a short period of time, for example, about ten minutes.

In another embodiment, there is disclosed a device for whitening teeth, consisting of a toothbrush with a head and a handle, where the head has a plurality of bristles, and at least one of the bristles contains a catalytic activator. The handle of this device has a reservoir for a bleaching compound and a means for dispensing the bleaching compound.

In a further embodiment, there is disclosed a device for whitening teeth, consisting of a toothbrush with a head and a handle, where the head has an applicator and a catalytic activator capable of catalyzing the reaction of a bleaching compound applied to the head.

### BRIEF DESCRIPTION OF THE DRAWINGS

FIG. 1 shows a side view of a toothbrush made in accordance with the present invention that has a compartment for a catalytic activator on the head.

FIG. 2 is a front elevational view thereof.

FIG. 3 shows a cross section of a toothbrush with bristles containing a catalytic activator.

### DETAILED DESCRIPTION OF SEVERAL EMBODIMENTS

All publications mentioned herein are incorporated herein by reference for the purpose of describing and disclosing the equipment, compositions, and methodologies which are described in the publications which might be used in connection with the presently described compositions and methods.

Disclosed herein is a composition for whitening teeth that includes a therapeutically effective amount of a bleaching compound and a catalytic agent, where the catalytic agent is capable of catalyzing the reaction of a significant portion of the bleaching compound within a short period of time. A “bleaching compound” is any compound which has the ability, when activated by a catalyst, of whitening the teeth. Examples of suitable bleaching compounds include an oxygen radical generating agent such as metal ion free peroxides, organic peroxides, and metal ion containing peroxides. Specific, non-limiting examples of bleaching agents suitable for use with the disclosed composition are carbamide peroxide, carbamyl peroxide, sodium percarbonate, perhydrol urea, and hydrogen peroxide. By “therapeutically effective amount” is meant the quantity of the bleaching agent, when placed in contact with a catalyst according to the present disclosure, necessary to whiten the teeth of a subject. A subject is any mammal, preferably a human.

Typically the bleaching agent is employed in the composition in amounts so that about 3% to 40% by weight comprises the bleaching agent. Preferably, if the bleaching compound is hydrogen peroxide, the concentration of the bleaching compound is from about 3 to 12% by weight. Most preferably, if the bleaching agent is hydrogen peroxide, the concentration of the bleaching compound is from about 6% to about 10% by weight. Preferably, if the bleaching agent is carbamide peroxide, carbamyl peroxide, sodium percarbonate, and perhydrol urea, the concentration of the bleaching compound is from about 10% to about 40% by weight. More preferably, if the bleaching agent is carbamide peroxide, carbamyl peroxide, sodium percarbonate or perhydrol urea, the concentration of bleaching compound is from about 20% to about 30% by weight.

Assays to determine a therapeutically effective amount of a bleaching compound are known in the art. For example, stained extracted teeth can be used to measure a whitening effect (see Example 1). Other assays, such as an analysis of the effect of a bleaching compound on the soft tissues, may also be used in the determination of a therapeutically effective range of concentrations of a bleaching compound.

The bleaching agent is contained in a vehicle; formulations of vehicles for bleaching agents are well known in the art. The formulation can be aqueous or non-aqueous. As an example, glycerin, and polyethylene glycol in combination with water are useful in formulating the vehicle. Thickening and gelling agents may also be used in the formulation of the bleaching agent. In one example, polyoxyethylene/polyoxypropylene block copolymers can be utilized. As another example, carbopol polymers can be used. These polymers are well known in the art, and are available commercially.

The composition further consists of a catalytic agent. A “catalytic agent” is a compound or molecule which accelerates the whitening action of the bleaching compound without being consumed in the reaction. In a preferred embodiment, the catalytic agent accelerates the release of oxygen radicals from an oxygen radical generating agent. Examples of such agents include, but are not limited to activated charcoal, platinum, platinum salts, copper, copper salts, silver, silver salts, and cobalt-containing compounds (particularly cobalt gluconate). In a preferred embodiment, activated charcoal or cobalt gluconate is used as the catalytic activator. Of particular use is the commercially activated charcoal Centaur, produced by Calgon, Inc. Catalytic agents catalyze the reaction of a significant portion of the bleaching compound within a short period of time of coming into contact with the bleaching compound. By “short period of time” is meant in minutes, preferably about ten minutes or less. The catalytic agent may catalyze the reaction of substantially all of the bleaching compound in about ten minutes of coming into contact with the bleaching agent. In one embodiment the catalytic activator catalyzes the reaction of 50% of the bleaching agent, preferably 70% of the bleaching agent, and more preferably 90% of the bleaching agent. Contact of the bleaching compound with the catalytic agent causes the visible evolution of gas within two minutes of contact. “Visible evolution” of a gas, such as oxygen, formed by the contact of the bleaching compound with the catalytic agent can be noted in many ways, such as the formation of bubbles or any sign of gaseous release.

In one embodiment, an abrasive material can be used in the composition. For example, a dicalcium phosphate abrasive may be incorporated into the composition (e.g., see U.S. Pat. No. 5,171,564). Example of dicalcium phosphate abrasives include, but are not limited to dicalcium phosphate dihydrate, anhydrous dicalcium, or calcium pyrophosphate. Other abrasives of use include siliceous materials. Examples of such materials include, but are not limited to, silica abrasives, such as precipitated amporphous hydrated silica, and alumina abrasives, such as alumina trihydrate, aluminum silicate, calcined alumina, and bentonite.

When an abrasive material is included the vehicle may contain water, humectant, surfactant, and a thickener. Examples of humectants are glycerin, sorbitol, and polyethylene glycol (molecular weight 200-1000). Both mixtures of humectants and single humectants can be employed in the composition. Thickeners may be incorporated in the abrasive component such as natural or synthetic gums such as carrageenan, xantham gum, sodium carboxymethyl cellulose, starch, polyvinylpyrrolidone, hydroxyethylpropylcellulose, hydroxybutyl methyl cellulose, hydroxypropyl methyl cellulose, and hydroxyethyl cellulose.

In another embodiment, an agent for administering fluoride, such as a fluorine providing salt, which has activity against cavities, may be incorporated into the composition. Such materials are characterized by their ability to release fluoride ions in water. Agents for administering fluoride include, but are not limited to, inorganic metal salts such as sodium fluoride, potassium, fluoride, and tin fluoride such as stannous fluoride and stannous chlorofluoride, sodium fluorosilicate, ammonium fluorosilicate, and sodium monofluorophosphate.

The composition may also include palliative ingredients for periodontal tissues. Examples of such ingredients include, but are not limited to, aloe, eugenol, and vitamin E. Pigments, sweeteners, colors, and flavors may also be incorporated into the composition. The composition may further include an ingredient to decrease tooth sensitivity, such as potassium nitrate. The addition of these compounds to dental creams, such as toothpaste, are well known in the art.

Also disclosed is a method for whitening teeth, by providing a bleaching agent, providing a catalytic agent, and then combining the bleaching agent with the catalytic agent, such that the catalytic agent catalyzes the reaction of a significant portion of the bleaching agent within about ten minutes of combining the bleaching agent with the catalytic agent.

Any convenient means can be utilized for separating the bleaching agent from the catalytic agent prior to the reaction process. In one embodiment, separate containers are used, one of which holds the bleaching agent in liquid form, and one of which holds the catalytic agent in solid or liquid form. The bleaching agent is then mixed with the catalytic agent immediately prior to use. For example, the bleaching agent can be in a liquid form, similar to a mouthwash. The catalytic agent can be in the form of a solid powder, contained in a foil packet. Just prior to use, the catalytic agent is added to the liquid containing the bleaching agent and the liquid is retained in the mouth, similar to a mouthwash, for three to five minutes.

In another embodiment, a single container can be compartmentalized so the bleaching agent is housed separately from the catalytic agent. Upon extrusion from the dual compartment container the bleaching agent is combined with the catalytic agent. For example, the whitening agent and the catalytic agents can each be contained in a gel or paste form. Upon extrusion from the container, the two are admixed, and placed on a toothbrush. Brushing for three to five minutes can also result in mixing the catalytic agent with the bleaching agent.

In another embodiment, a stable composition is formed consisting of the catalyst microencapsulated and mixed with the bleaching agent. Techniques for microencapsulation of individual components for tooth and gum dentifrice are well known in the art (see U.S. Pat. No. 5,403,578). The encapsulated material is stable until contacted with water. In this embodiment it is preferable for the base composition of the paste to be free of water. Upon use of the composition, and the addition of water, the bleaching agent is placed in contact with the catalyst.

Either a paste or gel containing the bleaching agent and the catalyst may be prepared. Alternatively, a fabric embedded with the bleaching agent and the catalyst can be produced. Upon the addition of water, thereby wetting the fabric, the bleaching agent and the catalyst are admixed.

In another embodiment, the catalytic agent can be applied to a solid surface, such as a dental tray (e.g., U.S. Pat. No. 4,990,089). The dental tray can be either pre-fabricated or customized. In order to produce a customized dental tray, a sheet of moldable plastic is used that will conform to a mold of an individual's teeth. Production of such trays is well known in the art. In brief: (1) a mold of the teeth to be whitened is prepared, (2) a sheet of plastic material of an appropriate size between 0.01 inch and 0.1 inch thick is obtained, (3) the plastic sheet is placed in a holder, and, if desired, silicone may be sprayed onto the plastic sheet, (4) the plastic sheet is heated until it becomes malleable, (5) the heated sheet is placed over the model, (6) the heated plastic is contoured to the mold, either manually or using a vacuum process, (7) the mold is removed, and (8) the plastic is trimmed. The catalytic agent may be embedded into a sponge which is inserted into the heated plastic. Alternatively, the catalytic agent can be embedded in a fabric which is bonded to the tray after the plastic has cooled. The catalytic agent can also be directly applied or bonded to the surface of the cooled plastic, such as by spraying the tray with a solution of copper salts. The bleaching agent is applied to the tray in the form of a paste or a liquid immediately prior to use, and the tray is then applied to the subject's teeth.

In another embodiment, the catalytic agent may be applied to the surface of a toothbrush. A “toothbrush” is a device designed for cleaning teeth that has a region for holding, such as a handle, and a region for cleaning, such as bristles or a sponge. The toothbrush is either manually or mechanically agitated to clean the teeth of a subject. The head of the brush can be made of any appropriate material, such as plastic, and can be designed to contain the catalyst, either by applying the catalytic agent directly to the head of the brush, either on the same side of the brush as the bristles or sponge, or on the opposite side of the brush. The catalytic agent can be embedded in a fabric that is bonded to the head of the brush, or the fabric embedded with the catalytic agent can be inserted into a compartment in the head of the brush.

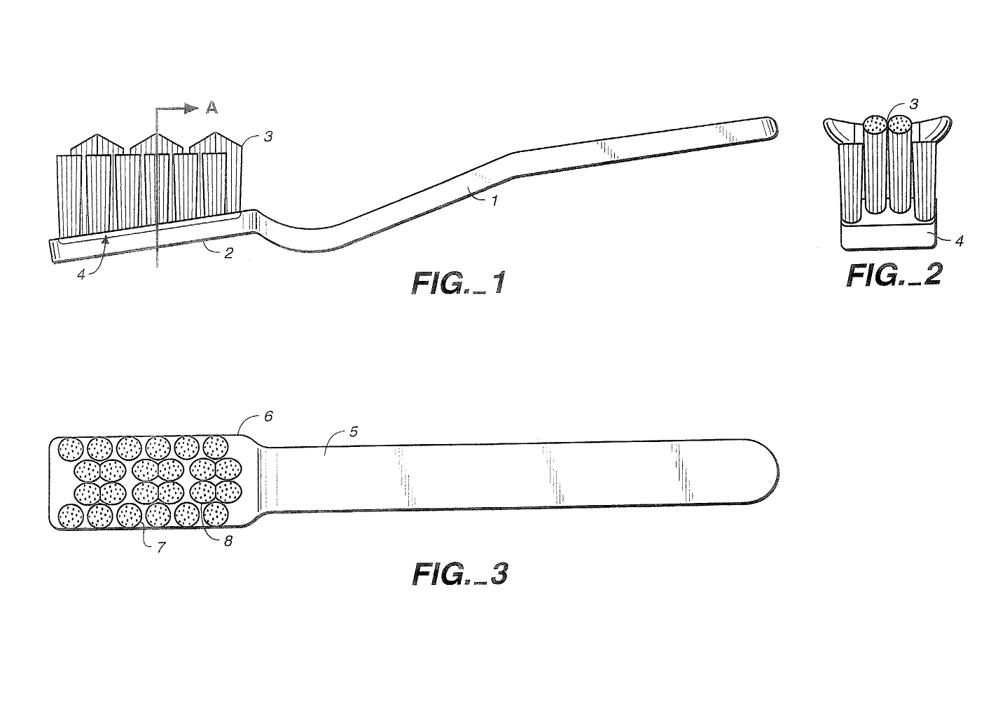

Referring to FIG. 1 there is shown a toothbrush, comprising a handle (1) and a head (2). The head (2) comprises bristles (3) and a compartment (4) for the insertion of a fabric or gel foam comprising a catalytic agent. The fabric or gel foam comprising the catalytic agent may be removed and replaced with a new piece of fabric or gel foam comprising a catalytic agent when desired.

A front elevational view of the device shown in FIG. 1, where a section is shown looking in the direction of (A), is depicted in FIG. 2. Referring to FIG. 2 it can be seen that the compartment (4) in the head of the brush is open to the upper surface of the brush. The bleaching compound is applied to the bristles prior to use of the toothbrush by the subject; the application of the bleaching compound to the toothbrush results in the catalyst contacting the bleaching compound. The subject then uses the toothbrush to brush the teeth, and the whitening effect is thereby achieved.

Alternatively, the bristles located on the head of the toothbrush can be designed to contain the catalyst: the catalyst can be applied to the surface of the bristle or it can be embedded into one or more of the bristles of the brush. Referring to FIG. 3, there is shown a toothbrush with a handle (5) and a head (6) with bristles (7). One of the bristles (8) is embedded with a catalytic agent. It should be noted that any number or conformation of bristles can be embedded with the catalytic agent. The bleaching compound is applied to the bristles prior to use of the toothbrush by the subject; the application of the bleaching compound to the toothbrush results in the catalyst contacting the bleaching compound. The subject then uses the toothbrush to brush the teeth, and the whitening effect is thereby achieved.

The following examples are intended to illustrate but not to limit the appended claims in any manner, shape or form, either explicitly or implicitly. While they are typical of those that might be used, other procedures, methodologies, or techniques known to those skilled in the art may alternatively be used.

### EXAMPLES

### Example 1

Stained extracted teeth were sectioned into two equal parts. Each of the pieces was submerged into a peroxide solution or gel. One piece of each tooth was submerged in peroxide alone, while the other was submerged in hydrogen peroxide with a catalyst, a hydrogen peroxide containing a modifier to raise the pH (either AMP or tris-Amino), or hydrogen peroxide containing both the catalyst and the modifier. Three solutions were tested: (1) 3% hydrogen peroxide, (2) 6% hydrogen peroxide, and (3) 10% hydrogen peroxide. Two gels were tested: (1) a commercially available dentist-supervised bleaching gel containing hydrogen peroxide up to 7%, and (2) a commercially available dentist-supervised bleaching gel containing carbamide peroxide up to 20%. Two catalysts were used in the experiments, either a standard activated charcoal or Centaur (Calgon) activated charcoal. Samples were photographed after three minutes of exposure, and at several other time points.

When a catalyst was used, significant bleaching was noted after twelve minutes. The bleaching was comparable to between 21 and 30 hours of bleaching without the catalytic agent. It was noted that those gels containing large amounts of carbopol provided the least amount of dental bleaching.

### Example 2

A catalytic agent was applied to a toothbrush by dipping a wet toothbrush into powdered catalytic agent, or by bonding activated charcoal to a toothbrush head between the bristles. A whitening gel (Natural White) containing 1.5% hydrogen peroxide was applied to the toothbrush and the brush was used to clean a subject's teeth. A whitening effect was noted after one application of three minutes in duration. One subject repeated the application of whitening gel in the presence of the catalytic agent twice a day for three days. Significant whitening was noted following three days of application.

### Example 3

Liquid 3% hydrogen peroxide was mixed with a few drops of mint flavor. A small amount of activated charcoal catalytic agent was placed into a cup, and one-half teaspoon of the peroxide was added. This solution was used to bathe the teeth of subjects for sessions of three minutes of duration, carried out for twice a day over a period of several days. Visible whitening was noted after two days.

It is to be understood that the disclosed compositions and methods are not limited to the particular methodology, and that protocols, models and reagents described as such may, of course, vary. It is also to be understood that the terminology used herein is for the purpose of describing particular embodiments only, and is not intended to limit the scope of the appended claims.

### Example 4

A specific formulation of the bleaching compound is as follows:

| Component | Concentration (percent by weight) |
|---|---|
| Hydrogen peroxide | 10% |
| Flavoring agent | 1% |
| aloe vera | 2% |
| Potassium nitrate | 1% |
| titanium dioxide | 1% |
| sodium lauryl sulfate | 1.5% |
| paste carrier | 83.5% |

In one embodiment, a catalyst is prepared consisting of activated charcoal and AMP mixed in the ration 2:1. The formulation comprising the bleaching compound and the catalyst are combined at the time of use in the ratio of 10:1.

In another embodiment, for use with a device disclosed herein, the formulation comprising the bleaching compound is placed on the toothbrush with a catalyst embedded into one or more of the bristles of the brush, or with a catalytic agent embedded in a fabric bonded to the head of the brush.

### Example 5

Two-part mixtures were prepared, referred to as part A and part B. A bleaching compound, sodium hydroxide and water were the ingredients used to form the following part A compositions:

6 wt. % hydrogen peroxide solution in water (referred to herein as “6% HP”);

12 wt. % hydrogen peroxide solution in water (referred to herein as “12% HP”); and

20 wt. % carbamide peroxide solution in water (referred to herein as “20% CP”).

Part B compositions according to the present disclosure were made by mixing 0.05 wt. % cobalt gluconate and deionized water. Comparative part B compositions were made by substituting manganese gluconate for the cobalt gluconate. Equal amounts of part A and part B were mixed together to provide a bath mixture. A control bath mixture that did not include any catalytic agent was also prepared.

Extracted human molars and enamel substitutes made from compressed calcium carbonate were both used as test substrates. The test substrates were stained and then placed in the various bath mixtures. The test substrates were removed from the bath mixture after 15 minutes.

The luminosity of each test substrate was measured prior to staining and immediately following removal from the bath mixture as described below. A positioning rig was made to hold the samples and a digital camera in a set position with multipoint full spectrum lighting providing even distribution of light to the samples. The samples were then photographed using the same f stop and exposure time for every sample, and the luminosity of the resulting image was analyzed using computer software (Adobe Photoshop). The average luminosity of all the samples tested in each bath mixture was calculated for each bath mixture. Luminosity was selected as a key measure of bleaching effectiveness because the human eye is significantly more sensitive to a change in luminosity compared to a change in hue or chroma (the other two aspects of color). Although a decrease in chroma is technically a result of bleaching, in the context of teeth a chroma reduction without a corresponding increase in luminosity, renders the teeth more gray rather than more white as perceived by the human eye. Moreover, an increase in teeth luminosity is perceived by the human eye as the teeth becoming whiter, even though the hue and chroma actually may have remained the same. Thus, the average initial luminosity was subtracted from the average final luminosity to arrive at a whitening score. The higher the whitening score, the greater the whitening effect observed.

The results showed a whitening score for the cobalt gluconate baths of 9.415 (6% HP), 25.06 (12% HP), and 11.67 (20% CP). The whitening scores for the comparative manganese gluconate baths were 5.385 (6% HP), 11.27 (12% HP), and 5.47 (20% CP). The whitening scores for the control baths were 3.69 (6% HP), 9.96 (12% HP), and 5.47 (20% CP). It is clear from these results that cobalt gluconate provided unexpectedly superior whitening.

What is claimed is:

1. A composition for whitening teeth, comprising a therapeutically effective amount of a bleaching compound and a catalytic activator, wherein said catalytic activator is selected from the group consisting of platinum, a platinum salt, palladium, a palladium salt, and a cobalt-containing compound and is capable of catalyzing the reaction of said bleaching compound within about ten minutes of contacting said catalytic activator with said bleaching compound.

2. The composition of claim 1, wherein the concentration of said bleaching compound is from about 3 to 40% by weight of the composition.

3. The composition of claim 2, wherein said bleaching compound is selected from the group consisting of carbamide peroxide, carbamyl peroxide, sodium percarbonate, perhydrol urea, and hydrogen peroxide.

4. The composition of claim 3, wherein said bleaching compound comprises hydrogen peroxide.

5. The composition of claim 1, further comprising an abrasive agent.

6. The composition of claim 1, further comprising an agent for administering fluoride.

7. The composition of claim 1, further comprising a gel forming agent.

8. The composition of claim 1, further comprising a palliative ingredient for periodontal tissues.

9. The composition of claim 8, wherein said palliative agent is selected from the group consisting of aloe, eugenol, and vitamin E.

10. The composition of claim 1, further comprising an ingredient to decrease tooth sensitivity.

11. The composition of claim 10, wherein said ingredient to decrease tooth sensitivity is potassium nitrate.

12. A composition for whitening teeth, comprising a therapeutically effective amount of a bleaching compound and a catalytic activator, wherein said catalytic activator comprises cobalt gluconate.

13. The composition of claim 12, wherein the concentration of said bleaching compound is from about 3 to 40% by weight of the composition.

14. The composition of claim 12, wherein said bleaching compound is selected from the group consisting of carbamide peroxide, carbamyl peroxide, sodium percarbonate, perhydrol urea, and hydrogen peroxide.

15. The composition of claim 12, wherein said bleaching compound comprises hydrogen peroxide.

16. A method for whitening teeth, comprising
applying to the teeth a composition that is prepared by combining a bleaching compound with a catalytic agent, wherein the catalytic agent comprises cobalt gluconate.

17. The method of claim 16, wherein the concentration of said bleaching compound is from about 3 to 40% by weight of the composition.

18. The method of claim 16, wherein said bleaching compound is selected from the group consisting of carbamide peroxide, carbamyl peroxide, sodium percarbonate, perhydrol urea, and hydrogen peroxide.

19. The method of claim 16, wherein said bleaching compound comprises hydrogen peroxide.

20. The method of claim 16, wherein the catalytic agent catalyzes the reaction of a significant portion of the bleaching agent within about ten minutes of contacting the teeth.

21. The method of claim 16, wherein the contact with the teeth occurs for at least three minutes.

22. A method for whitening teeth, comprising
applying to the teeth a whitening composition that includes a bleaching compound; and
accelerating the whitening action of the whitening composition by mixing a catalytic agent with the bleaching compound, wherein the catalytic agent is selected from the group consisting of platinum, a platinum salt, palladium, a palladium salt, silver, a silver salt, and a cobalt-containing compound.

23. The method of claim 22, wherein the concentration of said bleaching compound is from about 3 to 40% by weight of the composition.

24. The method of claim 22, wherein said bleaching compound is selected from the group consisting of carbamide peroxide, carbamyl peroxide, sodium percarbonate, perhydrol urea, and hydrogen peroxide.

25. The method of claim 22, wherein said bleaching compound comprises hydrogen peroxide and the cobalt-containing compound comprises cobalt gluconate.

26. A method for whitening teeth, comprising
applying to the teeth a whitening composition that includes a bleaching compound; and
catalyzing the reaction of the bleaching compound by mixing a catalytic agent with the bleaching compound, wherein the catalytic agent is selected from the group consisting of platinum, a platinum salt, palladium, a palladium salt, silver, a silver salt, and a cobalt-containing compound.

27. The method of claim 22, wherein the concentration of said bleaching compound is from about 3 to 40% by weight of the composition.

28. The method of claim 22, wherein said bleaching compound is selected from the group consisting of carbamide peroxide, carbamyl peroxide, sodium percarbonate, perhydrol urea, and hydrogen peroxide.

29. The method of claim 22, wherein said bleaching compound comprises hydrogen peroxide and the cobalt-containing compound comprises cobalt gluconate.

30. The method of claim 22, wherein the catalyzing of the reaction of the bleaching compound results in the evolution of a gas.

31. A method for whitening teeth, comprising
providing a first container that contains a bleaching compound;
providing a second container that contains a catalytic agent selected from the group consisting of platinum, a platinum salt, palladium, a palladium salt, silver, a silver salt, and a cobalt-containing compound; and
mixing the bleaching compound and the catalytic agent immediately prior to applying the resulting mixture to teeth.

32. The method of claim 31, wherein the concentration of said bleaching compound is from about 3 to 40% by weight of the composition.

33. The method of claim 31, wherein said bleaching compound is selected from the group consisting of carbamide peroxide, carbamyl peroxide, sodium percarbonate, perhydrol urea, and hydrogen peroxide.

34. The method of claim 31, wherein said bleaching compound comprises hydrogen peroxide and the cobalt-containing compound comprises cobalt gluconate.

35. A device for whitening teeth, comprising a compartmentalized container, wherein one compartment contains a therapeutically effective amount of a bleaching compound, and the other compartment contains a catalytic agent, wherein said catalytic agent is selected from the group consisting of platinum, a platinum salt, palladium, a palladium salt, silver, a silver salt, and a cobalt-containing compound.
